(19) United States
(12) Patent Application Publication
(10) Pub. No.: US 2008/0064120 A1
(43) Pub. Date: Mar. 13, 2008

(54) Raman spectroscopic lateral flow test strip assays

(76) Inventors: Richard H. Clarke, Big Sky, MT (US); M. Edward Womble, Austin, TX (US)

Correspondence Address: PATTON BOGGS LLP, 8484 WESTPARK DRIVE, SUITE 900, MCLEAN, VA, 22102, US

(21) Appl. No.: 11/698,083

(22) Filed: Jan. 26, 2007

Related U.S. Application Data
(63) Continuation-in-part of application No. 60/765,699, filed on Feb. 6, 2006.

(51) Int. Cl. G01N 33/558; G01N 21/65

(52) U.S. Cl. 436/514; 422/82.05

(57) ABSTRACT

The invention provides improved Raman spectroscopy-based methods and systems for the quantitative analysis of selected analytes using lateral flow binding assay test strips.

This Application claims the benefit of U.S. Ser. No. 60/765,699 filed Feb. 6, 2006, which is incorporated by reference herein in its entirety

### FIELD OF THE INVENTION

The invention relates generally to diagnostic assays, and, more particularly to lateral flow test strip assays and Raman spectroscopy.

### BACKGROUND OF INVENTION

A number of manufacturers market test kits based on an antibody immunoassay in which an analyte antigen is detected by binding to an antibody attached to a gold particle and subsequently detected by lateral flow of the sample, depositing the gold antibody-antigen complex at a test stripe location on the strip that presents a capture reagent, for example a second antibody that binds the analyte antigen at a different epitope as the first antibody. When a subject analyte is present in a test sample, the test stripe turns color, typically changing to red or pink, due to the coalescing of the gold particles at the test stripe into a sol-type solid that reflects light. The test is normally a qualitative test, good for identifying the presence of the antigen, but not providing any quantitative information.

The most well-known example of such a test kit is a home pregnancy test kit, designed to give either a positive or negative reading. Many other such test kits operate on the same principal (nicotine, viruses, drugs, bacterial infections) and may be made for any fluid analysis (blood, urine, saliva, etc). Marketers of these test kits include PolyMedCo, Meridian Diagnostics, Craig Medical and others. None of these products provide a quantitative answer, even though in many cases, the concentration of the antigen is important, as in the case of the pregnancy test in which the level of the hCG antigen is indicative of the stage of a pregnancy.

U.S. Pat. Nos. 5,376,556, 5,266,498, 5,445,972 and 5,567,628, each of which is incorporated by reference herein in its entirety, each describe a quantitative, lateral flow test strip system in which the beads/particles have a separate Raman-active label, in addition to an analyte-binding element, such as an antibody.

U.S. Pat. No. 6,514,767, which is incorporated by reference herein in its entirety, describes the preparation of beads having encapsulated Raman tags or “bar codes,” to which other molecules can be conjugated.

U.S. Pat. No. 6,750,065, which is incorporated by reference herein in its entirety, describes immunoassays involving surface-enhanced Raman scattering that are based on the displacement of a Raman label molecule.

U.S. Pat. No. 6,844,200, which is incorporated by reference herein in its entirety, describes devices for carrying out lateral-flow assays involving more than one analyte that are not based on Raman spectroscopy.

U.S. Pat. No. 6,924,153, which is incorporated by reference herein in its entirety, describes quantitative lateral flow assays and devices that are not based on Raman spectroscopy.

United States Publication No. 200502500141, which is incorporated by reference herein in its entirety, describes multiplex lateral flow immunoassays that utilize a quantum dot-based labeling and detection system which are not based on Raman spectroscopy.

### SUMMARY OF INVENTION

The invention provides a quantitative, Raman spectroscopy-based, lateral flow test strip assay system that is simplified in comparison to the lateral test flow assays known in the art, yet robust. In particular, the lateral flow test assays of the invention eliminate the use of a separate Raman label (or “tag”) for detection while providing quantitative assay results information. For example, in the present invention, the Raman signal relied upon for detection and quantification of the analyte arises from the analyte-binding element, which may be an antibody, which is bound to the bead members of the system and/or from the complex of the analyte-binding element and analyte, but not from a separate Raman label.

One embodiment of the invention provides a method for measuring at least one selected analyte in a liquid sample, that includes the steps of:

providing a lateral flow test strip unit that includes: (i) a sample zone for depositing a liquid biological sample, such as a body fluid, wherein the sample zone includes migratable particles, such as SERS-active particles, coated or otherwise bound with a first binding element, such as an antibody or aptamer, that is specific for an analyte to be measured, (ii) a capture zone having a second binding element, such as an antibody or aptamer, that specifically binds to the analyte and/or epitopes presented by the analyte and bound first binding element, but not by the first binding element alone; and

depositing a sample on the sample zone; and

after allowing sufficient time for migration of the particles through lateral flow strip, determining the presence, absence or concentration of the analyte in the sample by:

irradiating at least part of the capture zone with monochromatic light to generate a Raman spectra from particles (including from the first binding element thereon) that may be captured in the capture zone,

measuring the intensity of at least part of the Raman spectra that is specific for the first binding element on particles that may have been captured in the capture zone,

calculating the presence/absence or concentration of the analyte in the sample at least partly based on the measured intensity.

Another embodiment of the invention provides a method for measuring at least one selected analyte in a liquid sample that includes the steps of:

providing a lateral flow test strip unit that includes: (i) a sample zone for depositing a liquid biological sample, such as a body fluid, wherein the sample zone includes, migratable particles, such as SERS-active particles, coated with or otherwise bound to a first binding element, such as an antibody or an aptamer, that is specific for an analyte to be measured; (ii) a capture zone having a second binding element, such as an antibody or an aptamer, that specifically binds to the analyte and/or epitopes presented by the analyte and bound first binding element, but not by the first binding element alone, and (iii) a control zone having a third binding element, such as an antibody or an aptamer, that binds to the first binding element, the control zone, wherein the capture zone is located between the sample zone and the control zone;

depositing a sample on the sample zone,

after allowing sufficient time for migration of the particles through lateral flow strip, determining the presence, absence or concentration of the analyte in the sample by:

irradiating at least part of the capture zone with monochromatic light to generate a Raman spectra from particles (including from the first binding element thereon) that may be captured in the capture zone,

measuring the intensity of at least part of the Raman spectra that is specific for the first binding element on particles that may have been captured in the capture zone,

irradiating at least part of the control zone with monochromatic light to generate a Raman spectra from particles that may be captured in the control zone,

measuring the intensity of at least part of the Raman spectra that is specific for the first binding element on particles that may have been captured in the control zone,

calculating the presence/absence or concentration of the analyte in the sample at least partly based on a ratio of the intensities measured for the capture zone and the control zone.

A further embodiment of the invention provides a system for performing lateral flow test strip assays using Raman spectroscopy that includes: a portable Raman reader unit; and a lateral flow assay test strip unit comprising migratable SERS-active particles to which at least one analyte binding element is bound, in which the Raman reader unit and test strip unit are mutually adapted to allignedly position the test strip unit with respect to the Raman reader unit for the reading of at least one test or control stripe of the strip by the reader. In one variation, the reader unit and test strip unit are mutually adapted to allignedly position the test strip for the reading of at least one test stripe and at least one control stripe of the strip by the reader unit.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 shows a portion of the Raman spectrum of the test stripe of a AccuClear pregnancy lateral flow test kit, processed with a sample containing hCG, showing the characteristic strong Raman peaks of the hCG antibody gold conjugate embedded in the lateral flow strip.

FIG. 2 shows the full Raman spectrum of the test stripe from which the spectrum shown in FIG. 1 was extracted.

FIG. 3 shows a lateral flow assay test strip having notches for aligning the test stripe and control stripes of the strip with the optical probe of a Raman reader unit.

FIG. 4 shows the lateral test strip of FIG. 3 alignedly positioned in the positioning mechanism of a Raman reader unit for reading of the test stripe by the optical probe of the reader unit.

FIG. 5 shows a cross section of the test strip of FIG. 4 positioned in the test strip-guiding rails of the reader unit.

### DETAILED DESCRIPTION

One aspect of the present invention provides a method to read lateral flow immunoassay test kits quantitatively for the amount of the antigen present in the sample, using Raman spectroscopy. For example, a Raman spectrum or part thereof may be analyzed from the test stripe and optionally control stripe of an hCG (human chorionic gonadotropin) lateral flow immunoassay pregnancy test kit, such as the AccuClear hCG pregnancy test kit (Inverness Medical Innovations, Inc.). The Raman peaks at the test stripe (which is seen visually as a reddish stripe on the strip) are unique to the gold antibody conjugate, and the peak heights are directly proportional to the concentration of the captured gold complex. The Raman peak heights provide a quantitative readout either as a direct reading of the analyte peak intensity or as a ratio to either the Control stripe or to an additional Raman feature on the strip. The concentration of an analyte in a sample may, thus, be determined.

The gold particles of the bead-antibody conjugate may enhance the Raman signal by the SERS effect. Whether or not the present invention is implemented for surface-enhanced or natural scattering intensity, it relies on the Raman signature of the analyte-binding complex (such as bead-antibody complex) without the addition of a special Raman label. Using control reagents a concentration calibration may be readily produced for comparison with the observed Raman peak intensities. Also, since the size of the gold particles can be controlled when preparing the gold conjugates, a size may be chosen to optimize the Raman effect for quantification. The present invention may also utilized beads of material other than gold. For example, beads made of other SERS-active materials such as silver, nickel, copper and/or cadmium may be used. SERS-active metallic particles may, for example, be solid metallic particles or particles that are at least partially coated with a SERS-active metal. SERS techniques and materials are described in U.S. Pat. Nos. 5,400,136 and 5,864,397 to Vo-Dinh, which are incorporated herein by reference in their entirety.

Any suitable sort of Raman spectroscope or Raman scattering detection system may be used according to the invention. For example, high-resolution Raman systems as well as low-resolution Raman systems may be used. Information about Raman spectral analysis can be found in U.S. Pat. No. 5,139,334, which is incorporated herein by reference in its entirety and which teaches a low resolution Raman analysis system for determining certain properties related to hydrocarbon content of fluids. The system utilizes a Raman spectroscopic measurement of the hydrocarbon bands and relates specific band patterns to a property of interest. U.S. Pat. No. 5,982,484, which is incorporated by reference herein in its entirety, teaches sample analysis using low resolution Raman spectroscopy. U.S. Pat. No. 6,208,887, which is incorporated herein by reference in its entirety, teaches a low-resolution Raman spectral analysis system for determining properties related to in vivo detection of samples based on a change in the Raman scattered radiation produced in the presence or absence of a lesion in a lumen of a subject. Additionally, U.S. Pat. No. 6,897,951 entitled “Probe Assemblies for Raman Spectroscopy,” U.S. Pat. No. 6,643,012 entitled “Apertureless near-field scanning Raman microscopy using reflection scattering geometry,” U.S. Pat. No. 6,095,982 entitled “Spectroscopic method and apparatus for optically detecting abnormal mammalian epithelial tissue,” U.S. Pub. No. 20040174520 entitled “Low resolution surface enhanced Raman spectroscopy on sol-gel substrates,” U.S. Pub. No. 20040204634 entitled “Raman spectroscopic monitoring of hemodialysis,” U.S. Pub. No. 20050171436 entitled “Raman spectroscopy for monitoring drug-eluting medical devices,” and U.S. Pub. No. 20050128476 entitled “Raman spectroscope” are each also incorporated by reference herein in their entireties.

### EXAMPLE

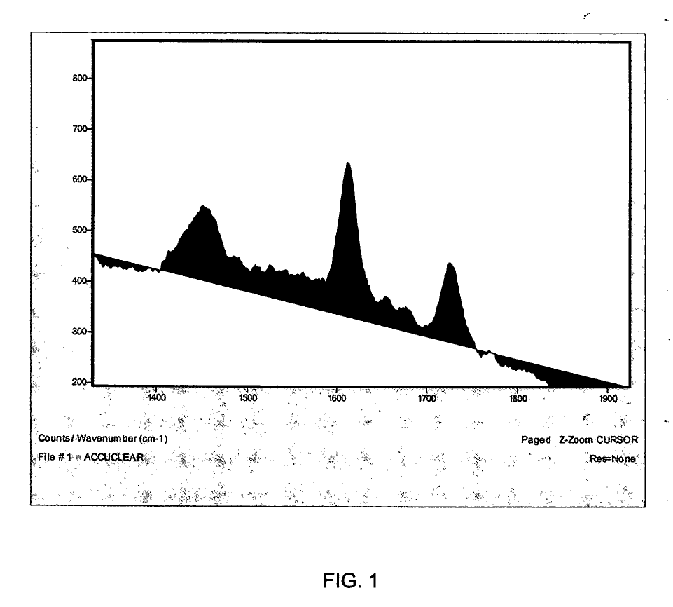

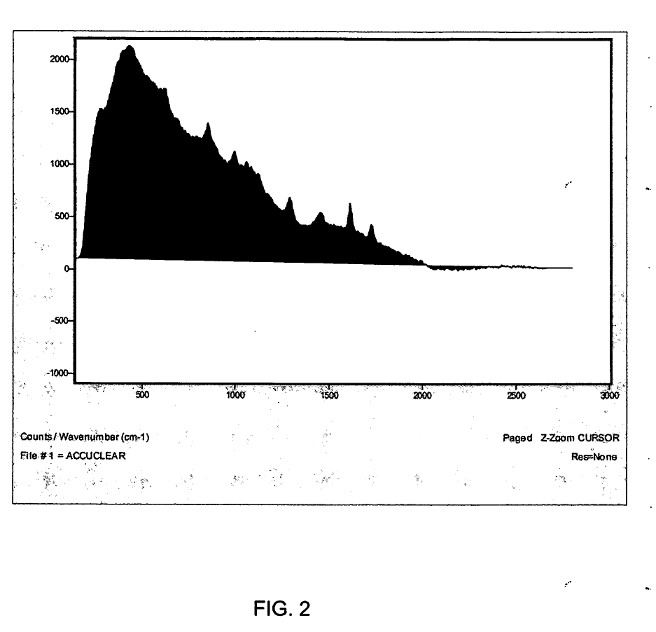

A direct implementation of the present invention may be seen in the spectrum obtained in the spectrum shown in FIGS. 1 and 2, which was obtained by focusing the probe of a RSI R-3000 Raman system (Raman Systems, Inc., Austin, Tex.) on an AccuClear hCG test kit and measuring either the peak intensity directly (after a suitable calibration run) or by ratioing the Raman peak intensities at the T (test) and C (control) positions on the test kit after the liquid sample has been allowed to flow laterally though the kit. FIG. 1 shows a portion of the Raman spectrum demonstrating the characteristic strong Raman peaks of the hCG antibody gold conjugate embedded in the lateral flow strip. FIG. 2 shows the full Raman spectrum of the test stripe from which the spectrum shown in FIG. 1 was extracted.

In one embodiment of the invention a portable lateral flow test strip Raman reader is provided that includes optics for illuminating the test and/or control strips of a lateral flow test strip with monochromatic light to generate a Raman signal, optics for collecting the signal, a Raman spectrometer for separating and quantifying at least some of the components of the Raman signal, and at least one computer processor linked to the spectrometer, and working in conjunction with memory, for analyzing information from the spectrometer to determine the presence, absence and/or concentration of a test analyte. The reader may, for example, be sized to be handheld. Where the system of the invention relies on detecting the Raman signal from a particle-bound, analyte-binding element such as an analyte-binding antibody, a high-resolution Raman spectroscopic apparatus may be used but is not necessary to quantify analyte in a test sample. Accordingly, a compact low-resolution Raman reader unit may be employed.

One embodiment of the invention provides a lateral flow assay test strip that is notched, has other physical elements and/or is marked to permit the operative alignment of the stripes with a Raman reader unit (Raman spectrometer) so that the test and/or control stripe(s) regions can be read. For example, the marking and/or notch(es) may be in register with the stripes or they can be offset from the stripes so long as the probe of the Raman reader unit is suitably positioned (coordinated) to read a/the strip when the mark or notch is correctly positioned. A related embodiment provides a Raman reader that includes a test strip receiving member adapted to align, or allow the alignment of, the test stripes with illuminating and signal receiving elements of the Raman spectrometer based on a reference marking and/or notche(s) present on a later flow assay test strip. A still further embodiment provides a system that includes the aforementioned test strip and a Raman reader unit that are adapted to be used together. The lateral flow matrix (material) may or may be at least partially housed in a casing. In this case, the casing, rather than the actual strip material may include one or more reference markings, notches and/or other physical elements that permit the strip to be properly aligned in the reader for reading of the stripe(s).

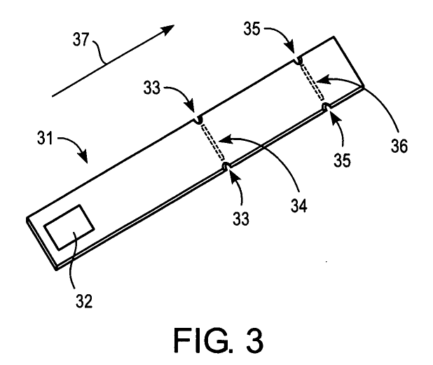

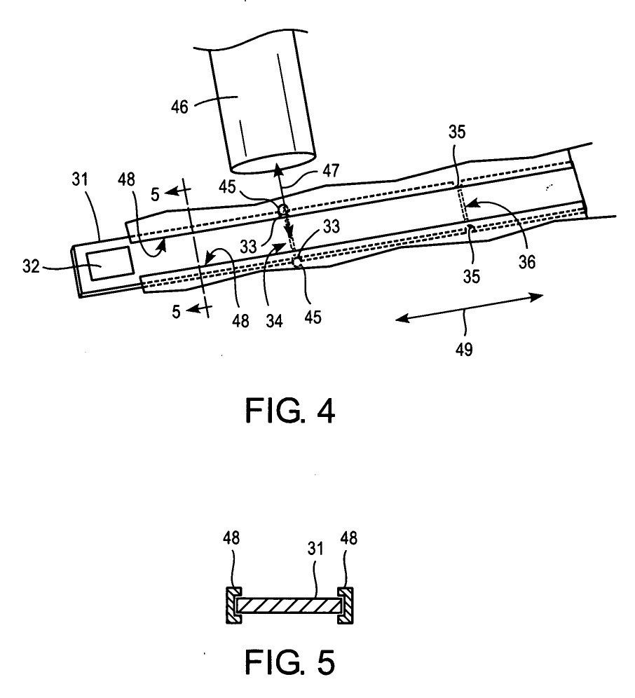

For example, FIG. 3 shows a lateral flow assay test strip 3 1, having a sample deposition area 32 and bilateral notches 33 formed at the axial position of a test stripe 34 and bilateral notches 35 formed at the position of a control stripe 36. Arrow 37 illustrates the direction of lateral flow in the test strip. FIG. 4 shows an example of the test strip of FIG. 3, positioned in a strip positioning mechanism of a Raman reader apparatus. Lateral protrusions 45 of the reader align with notches 33 of the test strip so the strip clicks into position for reading of the test stripe (as currently shown) or control stripe by the Raman probe 46 to illuminate test stripe 34 and collect resulting Raman scatter light therefrom, along optical path 47. The test strip 31 and/or protrusions 45 have sufficient give or springiness so that the test strip can be clicked from one alignment position into another by an operator. Guide rail “tongues” 48 of the reader unit overlay the edges of the test strip and collectively form a slot in which the test strip is guided. Arrow 49 shows the lateral direction in which the test strip can be moved forward and backwards. In one embodiment, the Raman reader unit prompts the operator to position the test strip at one or more test stripes and/or control stripes. FIG. 5 shows a cross sectional view, along line 5 of FIG. 4, of the test strip positioned in the test strip-guiding rails of the reader unit.

Raman intensity (peak height) is directly proportional to the concentration of a scatterer. Accordingly, the concentration of an analyte may, for example, be determined by multiplying the Raman intensity at a wavenumber or wavenumber band associated with the Raman scattering of the bead-bound analyte-binding element (which may be an antibody), or a complex or the analyte-binding element and analyte, at the test stripe for the analyte, with a proportionality constant, which may, for example, be determined in advance using control samples having known concentrations of a subject analyte. For embodiments in which the test strip also includes a control stripe, a ratio of Raman signal readings from the test stripe and control stripes may, for example, be multiplied with a proportionality constant to obtain the desired analyte concentration. Using such a ratio advantageously corrects for potential calibration variabilities that may arise with the Raman reader unit.

Fluid and liquid samples that may be assayed according to the invention include, but are not limited to, blood, urine and saliva. Other body fluids that may be assayed include, for example, lymph and cerebrospinal fluid. Body fluids that are assayed may be unprocessed (“raw”), such as blood, or processed, such as plasma. Semi-fluids such as sputum or fecal matter may also be assayed. Non-fluid or semi-fluid samples may be also fluidized or further fluidized for assay according to the invention.

Antibodies used as binding elements may be of any suitable form or type, may be produced by any method, and may be unprocessed or processed, for example proteolytically into FAb fragments.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A method for measuring at least one selected analyte in a liquid sample, comprising the steps of:
providing a lateral flow test strip unit that includes:
a sample zone for depositing a liquid biological sample, such as a body fluid, wherein the sample zone includes, migratable SERS-active particles, coated with a first binding element, that is specific for an analyte to be measured,
a capture zone having a second binding element that specifically binds to the analyte and/or epitopes presented by the analyte and bound first binding element, but not by the first-binding element alone; and
depositing a sample on the sample zone; and
after allowing sufficient time for migration of the particles through lateral flow strip, determining the presence, absence or concentration of the analyte in the sample by:
irradiating at least part of the capture zone with monochromatic light to generate a Raman spectra from particles that may be captured in the capture zone,
measuring the intensity of at least part of the Raman spectra that is specific for the first binding element on particles that may have been captured in the capture zone,
calculating the presence/absence or concentration of the analyte in the sample based on the measured intensity.

2. The method of claim 1, wherein at least one of the first and second binding elements is an antibody.

3. The method of claim 1, wherein the SERS-active particles comprise gold.

4. The method of claim 1, wherein the SERS-active particles are colloidal gold particles.

5. The method of claim 1, wherein the step of determining the presence, absence or concentration of the analyte in the sample further comprises inserting the lateral flow test strip unit into a portable Raman analyzer unit adapted to receive said test strip unit for reading.

6. The method of claim 5, wherein the test strip unit and the Raman analyzer unit are mutually adapted to position the test strip for reading by the Raman analyzer unit.

7. The method of claim 5, wherein the portable Raman analyzer unit comprises an excitation light source, a Raman spectroscope and a processor operably linked to the Raman spectroscope to determine the presence, absence or concentration of the analyte in the sample based, at least in part, on the intensity of the Raman signal of the first binding elements associated with the particles that are captured in the capture zone.

8. The method of claim 7, wherein the analyzer unit further comprises a display that is operably linked to the processor.

9. The method of claim 7, wherein the Raman analyzer unit is a low-resolution Raman spectroscopy unit.

10. A method for measuring at least one selected analyte in a liquid sample, that includes the steps of:
providing a lateral flow test strip unit that includes,
a sample zone for depositing a liquid biological sample, such as a body fluid, wherein the sample zone includes migratable SERS-active particles coated with a first binding element, that is specific for an analyte to be measured
a capture zone having a second binding element that specifically binds to the analyte and/or epitopes presented by the analyte and bound first binding element, but not by the first binding element alone, and
a control zone having a third binding element such as an antibody that binds to the first binding element, the control zone, wherein the capture zone is located between the sample zone and the control zone;
depositing a sample on the sample zone,
after allowing sufficient time for migration of the particles through lateral flow strip, determining the presence, absence or concentration of the analyte in the sample by:
irradiating at least part of the capture zone with monochromatic light to generate a Raman spectra from particles that may be captured in the capture zone,
measuring the intensity of at least part of the Raman spectra that is specific for the first binding element on particles that may have been captured in the capture zone,
irradiating at least part of the control zone with monochromatic light to generate a Raman spectra from particles that may be captured in the control zone,
measuring the intensity of at least part of the Raman spectra that is specific for the first binding element on particles that may have been captured in the control zone.

11. The method of claim 10, further comprising the step of:
calculating the presence/absence or concentration of the analyte in the sample based on a ratio of the intensities measured for the capture zone and the control zone.

12. The method of claim 10, wherein at least one of the first, second and third binding elements is an antibody.

13. A system for performing lateral flow test strip assays using Raman spectroscopy, comprising:
a portable Raman reader unit; and
a lateral flow assay test strip unit comprising migratable SERS-active particles to which at least one analyte binding element is bound,
wherein the Raman reader unit and test strip unit are mutually adapted to allignedly position the test strip unit with respect to the Raman reader unit for the reading of at least one test or control stripe of the strip by the reader unit.

14. The system of claim 13, wherein at least one test or control stripe of the strip comprises at least one test stripe and at least one control stripe of the strip.
